(19) United States
(12) Patent Application Publication
(10) Pub. No.: US 2012/0252856 A1
(43) Pub. Date: Oct. 4, 2012

(54) PI3K/AKT PATHWAY SUBGROUPS IN CANCER: METHODS OF USING BIOMARKERS FOR DIAGNOSIS AND THERAPY

(75) Inventors: Anna Joy, Phoenix, AZ (US); Burt G. Feuerstein, Phoenix, AZ (US); Ivan Smimov, Greenbrae, CA (US)

(73) Assignee: Dignity Health, Phoenix, AZ (US)

(21) Appl. No.: 13/515,494

(22) PCT Filed: Dec. 10, 2010
(86) PCT No.: PCT/US10/59953

§ 371 Date: Jun. 12, 2012

Related U.S. Application Data
(60) Provisional application No. 61/285,936, filed on Dec. 11, 2009.

(51) Int. Cl. A61K 31/4188; A61P 35/00; G01N 33/53; C12Q 1/68; C40B 30/00

(52) U.S. Cl. 514/393; 435/6.12; 506/7; 436/501; 435/7.94; 435/7.9

(57) ABSTRACT

The present invention relates to methods of dividing cancer into subgroups based upon Akt pathway gene expression. In one embodiment, the present invention provides a method of diagnosing glioblastoma multiforme (GBM) subtype in an individual by determining the presence of an abnormal expression of an Akt pathway gene cluster and diagnosing the cancer subtype in the individual.

### FIELD OF THE INVENTION

The invention relates to the field of biotechnology; specifically, to cancer diagnostics and therapy related to the Akt pathway and other cell death related pathways.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Standard therapies treat GBM as one disease, but variations in natural history and therapeutic response indicate it is not. Molecular profiling suggests that there could be molecular subtypes. Failure to classify GBM subtype can affect patient treatment, drug development and clinical trials. Clinical trials that do not stratify for subgroups will be underpowered and could miss subtype-specific drugs. Furthermore, unstratified patients may bear extra expense and toxicity. Targets within a subgroup might be missed if GBM are considered as a whole. The PI3K/Akt pathway is one of the 3 core pathways consistently altered in GBM. It often leads to activation of Akt. Akt is an oncogenic serine/threonine kinase that regulates metabolism, survival, autophagy, proliferation, migration, epithelial to mesenchymal (EMT) transition and angiogenesis. The pathway is a large and complex with many regulators, activators, effectors and feedback loops. It is not known if all GBM or other classes of tumors use the Akt pathway similarly.

Thus, there is a need in the art for novel biomarkers and/or genetic markers for GBM subgroups, specifically in Akt pathway gene expression and survival, as well as further pathway analysis within subgroups to identify subgroup-specific targets.

### SUMMARY OF THE INVENTION

Various embodiments include a method of diagnosing a cancer subtype in an individual, comprising determining the presence or absence of an abnormal expression of an Akt pathway gene cluster in the individual, and diagnosing the cancer subtype based on the presence of the abnormal expression of the Akt pathway gene cluster in the individual. In another embodiment, the cancer is glioblastoma multiforme (GBM). In another embodiment, the Akt pathway gene cluster is generated from one or more genetic loci listed in FIG. 7 herein. In another embodiment, the abnormal expression of an Akt pathway gene cluster comprises an overexpression of PDGFR\acute{α} and/or EGFR in the individual. In another embodiment, the individual is a human. In another embodiment, the individual is a mouse and/or rat. In another embodiment, the Akt pathway gene cluster comprises one or more of the following genetic loci: SORBS, PPP2R2C, TP53, PIK3C3, FGFR3, PPP2R5B, Akt1, Akt1S1, HIF1A, EIF4EBP1, EGFR, PDGFC, PDGFA, PHLPP, PDGFRA, RICTOR, AKT1P, TWIST, CCND1, MDM2, GAB2 and/or HSP90B 1. In another embodiment, the abnormal expression of the Akt pathway gene cluster comprises a high level of expression relative to a normal subject of SORBS, PPP2R2C, TP53, PIK3C3, FGFR3, PPP2R5B, Akt1, Akt1S1, HIF1A, EIF4EBP1, EGFR, PDGFC, PDGFA, PHLPP, PDGFRA, RICTOR, AKT1P, TWIST, CCND1, MDM2, GAB2 and HSP90B1, or any combinations thereof.

Other embodiments include a method of treating cancer in an individual, comprising diagnosing a cancer subtype in the individual based on a cluster of Akt pathway gene expression, and treating the individual. In another embodiment, the cluster of Akt pathway gene expression is generated from one or more genetic loci listed in FIG. 7 herein. In another embodiment, treating the individual comprises administering a therapeutically effective dosage of temodar (TMZ) to the individual. In another embodiment, treating the individual comprises administering a therapeutically effective dosage of PDGFR\acute{α} inhibitor to the individual. In another embodiment, treating the individual comprises administering a therapeutically effective dosage of EGFR inhibitor to the individual. In another embodiment, the cluster of Akt pathway gene expression comprises one or more of the following genetic loci: SORBS, PPP2R2C, TP53, PIK3C3, FGFR3, PPP2R5B, Akt1, Akt1S1, HIF1A, EIF4EBP1, EGFR, PDGFC, PDGFA, PHLPP, PDGFRA, RICTOR, AKT1P, TWIST, CCND1, MDM2, GAB2 and/or HSP90B1. In another embodiment, the cluster of Akt pathway gene expression comprises a high level of expression relative to a normal subject of SORBS, PPP2R2C, TP53, PIK3C3, FGFR3, PPP2R5B, Akt1, Akt1S1, HIF1A, EIF4EBP1, EGFR, PDGFC, PDGFA, PHLPP, PDGFRA, RICTOR, AKT1P, TWIST, CCND1, MDM2, GAB2 and HSP90B1, or any combinations thereof. In another embodiment, diagnosing the cancer subtype based on the cluster of Akt pathway gene expression comprises protein analysis, polypeptide modification, polynucleotide modification, gene mutation analysis, and/or gene sequencing. In another embodiment, the cancer is glioblastoma multiforme (GBM).

Other embodiments include a method of diagnosing a tumor subtype, comprising obtaining a tumor sample from an individual, assaying the tumor sample to determine the presence or absence of an abnormal expression of an Akt pathway gene cluster, and diagnosing the tumor subtype based on the presence of the abnormal expression of the Akt pathway gene cluster. In another embodiment, the abnormal expression of the Akt pathway gene cluster comprises a high level of expression relative to a normal subject of SORBS, PPP2R2C, TP53, PIK3C3, FGFR3, PPP2R5B, Akt1, Akt1S1, HIF1A, EIF4EBP1, EGFR, PDGFC, PDGFA, PHLPP, PDGFRA, RICTOR, AKT1P, TWIST, CCND1, MDM2, GAB2 and HSP90B1, or any combinations thereof. In another embodiment, the tumor comprises glioblastoma multiforme (GBM). In another embodiment, the Akt pathway gene cluster comprises any biomarker including but not limited to nucleic acids, proteins, modified proteins, mutated or modified nucleic acids, epigenetic changes or an associated change in DNA copy number.

Other features and advantages of the invention will become apparent from the following detailed description, taken in conjunction with the accompanying drawings, which illustrate, by way of example, various embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts, in accordance with an embodiment herein, a plot of correlations between clustered samples. (A) Expression profiling of 14 non-neoplastic autopsy specimens from donors with no history of brain tumor or neurological disorder and 181 HGG was performed using Affymetrix U133A and U133B chips on tumors collected at UCSF, MDA, and UCLA (GSE4271 and GSE4412). A sample correlation cluster map was generated using a hand curated list of Akt pathway genes. (B) Kaplan Meier curves for tumors in clusters 1 through 5 and nonclustering tumors. (C) Differences in Kaplan Meier survival curves for patients in subgroups 4 and 5 approach significance; p=0.06 (log rank). Results: There are 5 subgroups of HGG patients that have different expression of Akt pathway genes and different survival curves. There are 3 well defined clusters of tumors, 2 less defined clusters, and a group of genes (lower left) that are not part of well defined clusters (cluster 0).

FIG. 2 depicts, in accordance with an embodiment herein, GBM tumors cluster into distinct subtypes based on expression of PI3K/Akt pathway genes. Expression profiling was as described in FIG. 1 herein. (A) Two way unsupervised hierarchical clustering was performed using Pearson/centroid metric/linkages for PI3K/Akt pathway genes in all tumors and non-neoplastic brain. Cluster numbers 1-5 (labeled at the bottom) contain tumors identified from the plot of correlations between clustered samples shown in FIG. 1. Results: PDGFRalpha is overexpressed in subgroup 4 and EGFR in subgroup 3, among other results.

FIG. 3 depicts, in accordance with an embodiment herein, Akt subgroups in GBM. Correlation map generated using Akt pathway genes and GSE4271 (expression profiling results from 171 WHO grade IV astrocytoma and 14 non-neoplastic controls from autopsy). Map generated with a custom program implemented in R (A). Similar results were obtained using the TCGA dataset (B). Kaplan Meyer curves are plotted for patient subgroups. Results: There are 5 patient subgroups that have different patterns of Akt pathway gene expression.

FIG. 4 depicts, in accordance with an embodiment herein, recurrent tumors fall in subgroups 0, 3, 4 and 5.

FIG. 5 depicts distribution of Akt pathway genes in subgroups. Two-way unsupervised hierarchical clustering was performed using Pearson/Centroid metric/linkages for Akt pathway genes in GSE4271 with nonclustering tumors removed. Tumors in clusters 1-5 correspond to clusters in FIG. 3.

FIG. 6 (prior art) depicts a schematic representation of the Akt pathway.

FIG. 7 depicts, in accordance with an embodiment herein, a list of genes that when used in clustering methods, may divide tumors into subgroups. The list includes genes by official symbol as well as their entrez gene ID number.

FIG. 8 depicts, in accordance with an embodiment herein, human-rodent xenograft models of Akt subgroups associated with TMZ sensitivity. The inventors analyzed replicates of 15 xenografts and 1 human cell line for Akt classes. Mean survival for placebo, temodar (TMZ), radiation (RT) or concurrent TMZ+RT treated mice in each subgroup (B). Significance determined with a 2-sample, 2-sided t test assuming unequal variance. Intracranial xenografts are prepared from flank passaged GBM tissue.

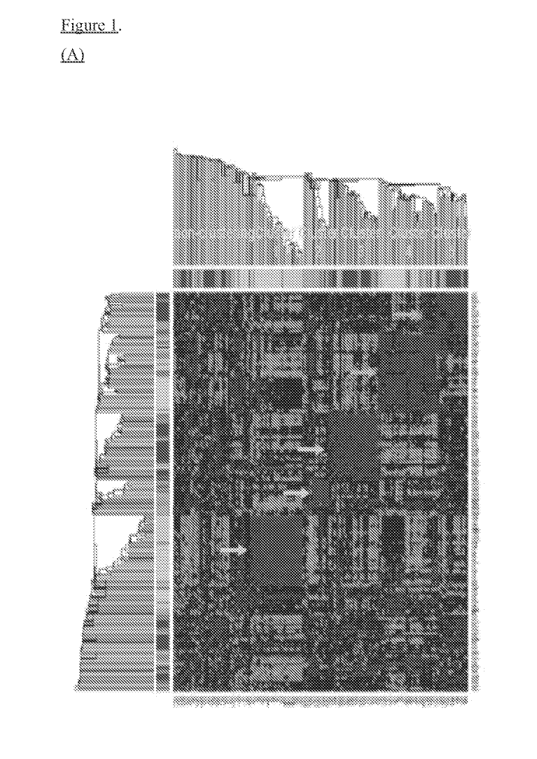

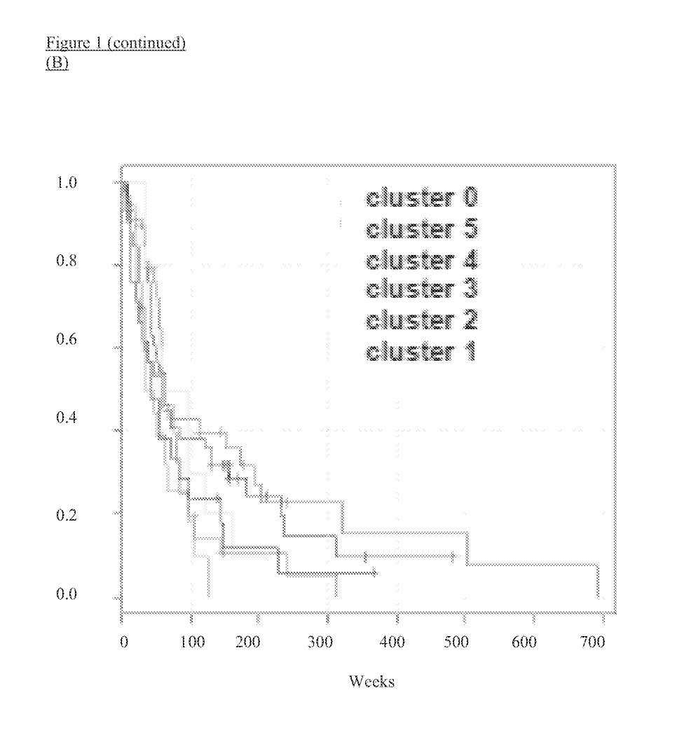

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

As used herein, the term “HGG” means high grade glioma.

As used herein, the term “GBM” means glioblastoma multiforme.

As used herein, the term “TMZ” means temodar.

As used herein, the term “RT” means radiation.

As disclosed herein, biomarkers that select patients for therapeutics would benefit clinical trial design and patient care. The Akt pathway is a therapeutic target in Glioblastoma Multiforme (GBM) and an important determinant of patient outcome. However, it is not known whether activity of this pathway varies among GBM tumors. To examine differences in AKT pathway among GBM, the inventors investigated mRNA expression of Akt pathway genes in published GBM expression datasets. It was found at least 5 distinct patterns of Akt pathway gene expression, and the patterns were prognostic. Pathway analysis suggests specific molecular targets within these Akt groups. Therefore Akt subgroups will help select patients for targeted therapies. Since Akt is an important determinant of response to conventional therapies, Akt subgroups will help select patients for conventional therapies.

As further disclosed herein, HGG tumors cluster into distinct subtypes based on expression of PI3K/Akt pathway genes. Cluster numbers 1-5 contain tumors identified from the plot of correlations between clustered samples, with PDGFRalpha overexpressed in subgroup 4 and EGFR in subgroup 3.

In one embodiment, the present invention provides a method of diagnosing a cancer subtype by detecting the presence or absence of an Akt pathway gene expression profile, where the presence of the Akt pathway gene expression profile is indicative of the cancer subtype. In another embodiment, the cancer subtype is associated with temodar (TMZ) sensitivity. In another embodiment, the cancer is glioblastoma multiforme and/or high grade glioma. In another embodiment, the Akt pathway gene expression profile is one of five possible clusters of gene expression profiles. In another embodiment, the Akt pathway gene expression profile is characterized by an overexpression of PDGFRalpha. In another embodiment, the Akt pathway gene expression profile is characterized by an overexpression of EGFR.

In one embodiment, the present invention provides a method of treating an individual for cancer by determining the presence of an Akt pathway gene expression profile or any other gene(s), protein(s), modified protein(s), nucleic acid(s), modified or mutated nucleic acid(s), epigenetic change(s), or DNA copy number changes associated with an Akt subgroup, and treating the individual. In another embodiment, the present invention provides a method of treating an individual for cancer by determining the presence of an abnormal activation of an Akt pathway, and treating the individual by administering the appropriate therapy. In another embodiment, the appropriate therapy is administering a therapeutically effective dosage of temodar (TMZ) or other antineoplastic agent to the individual.

In another embodiment, the present invention provides a method of treating a cancer subtype by diagnosing an Akt pathway gene expression profile characterized by overexpression of PDGFRalpha, and then treating the cancer by administering a therapeutically effective dosage of PDGFRalpha inhibitors. In another embodiment, the present invention provides a method of treating a cancer subtype by diagnosing an Akt pathway gene expression profile characterized by overexpression of EGFR, and then treating the cancer by administering a therapeutically effective dosage of EGFR inhibitors.

Analysis of the nucleic acid from an individual, whether amplified or not, may be performed using any of various techniques readily available and apparent to one of skill in the art. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

Similarly, there are many techniques readily available in the field for detecting the presence or absence of polypeptides or other biomarkers, including protein microarrays. For example, some of the detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Similarly, there are any number of techniques that may be employed to isolate and/or fractionate biomarkers. For example, a biomarker may be captured using biospecific capture reagents, such as antibodies, aptamers or antibodies that recognize the biomarker and modified forms of it. This method could also result in the capture of protein interactors that are bound to the proteins or that are otherwise recognized by antibodies and that, themselves, can be biomarkers. The biospecific capture reagents may also be bound to a solid phase. Then, the captured proteins can be detected by SELDI mass spectrometry or by eluting the proteins from the capture reagent and detecting the eluted proteins by traditional MALDI or by SELDI. One example of SELDI is called “affinity capture mass spectrometry,” or “Surface-Enhanced Affinity Capture” or “SEAC,” which involves the use of probes that have a material on the probe surface that captures analytes through a non-covalent affinity interaction (adsorption) between the material and the analyte. Some examples of mass spectrometers are time-of-flight, magnetic sector, quadrupole filter, ion trap, ion cyclotron resonance, electrostatic sector analyzer and hybrids of these.

Alternatively, for example, the presence of biomarkers such as polypeptides maybe detected using traditional immunoassay techniques. Immunoassay requires biospecific capture reagents, such as antibodies, to capture the analytes. The assay may also be designed to specifically distinguish protein and modified forms of protein, which can be done by employing a sandwich assay in which one antibody captures more than one form and second, distinctly labeled antibodies, specifically bind, and provide distinct detection of, the various forms. Antibodies can be produced by immunizing animals with the biomolecules. Traditional immunoassays may also include sandwich immunoassays including ELISA or fluorescence-based immunoassays, as well as other enzyme immunoassays.

Prior to detection, biomarkers may also be fractionated to isolate them from other components in a solution or of blood that may interfere with detection. Fractionation may include platelet isolation from other blood components, sub-cellular fractionation of platelet components and/or fractionation of the desired biomarkers from other biomolecules found in platelets using techniques such as chromatography, affinity purification, 1D and 2D mapping, and other methodologies for purification known to those of skill in the art. In one embodiment, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Generally

As disclosed herein, biomarkers that select patients for therapeutics would benefit clinical trial design and patient care. The Akt pathway is a therapeutic target in Glioblastoma Multiforme (GBM) and an important determinant of patient outcome. However, it is not known whether activity of this pathway varies among GBM tumors. To examine differences in AKT pathway among GBM, the inventors investigated mRNA expression of Akt pathway genes in published GBM expression datasets. It was found at least 6 distinct patterns of Akt pathway gene expression, and the patterns were prognostic. Pathway analysis suggests specific molecular targets within these Akt groups. Therefore Akt subgroups will help select patients for targeted therapies. Since Akt is an important determinant of response to conventional therapies, Akt subgroups will help select patients for conventional therapies.

### Example 2

### Discussion

The inventors show there are at least 6 classes of GBM with different survival and patterns of Akt pathway gene expression. Survival differences suggest Akt class predicts either prognosis (tumor aggressiveness independent of therapy) or response to therapy. The Akt pathway is a partial determinant of sensitivity to both conventional and targeted therapies. Therefore the inventors believe that Akt class predicts response to conventional and targeted therapies. Other data supports this. EGFR and PDGFRα are established therapeutic targets in GBM. mRNA for these receptors is differentially expressed in subgroups. This supports Akt class can predict response to therapeutics targeting these receptors.

The inventors perform gene set enrichment analysis (GSE) using all profiled genes to find pathways activated in subgroups. The data showing EGFR and PDGFRα mRNA are subgroup specific and suggest different pathways are activated in subgroups. This supports mining subgroups for pathways will enhance target identification.

### Example 3

### Conclusions

There are at least 6 classes of GBM with different patterns of Akt pathway gene expression. Furthermore, it demonstrates that Akt class can be used to match therapy to patient. Additionally, mining of Akt classes will enhance identification of subgroup-specific targets.

### Example 4

### The PI3K/Akt Pathway is an Important Therapeutic Target in High Grade Glioma (HGG) and Many Other Cancers

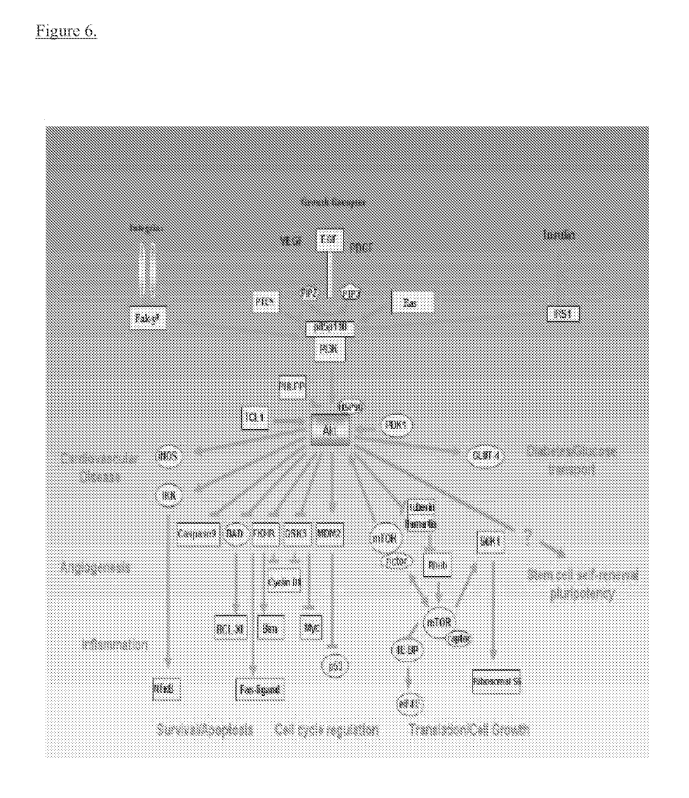

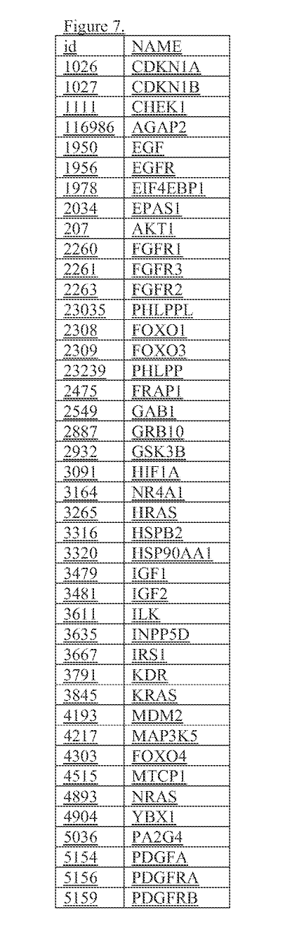

Akt is an oncogenic serine/threonine kinase that is a key effector in the PI3K/Akt pathway. This large and complex pathway regulates many functions important in cancer including migration, angiogenesis, proliferation, epithelial to mesenchyme transition (EMT) stem cell self-renewal and resistance to cytotoxic therapy [1-5]. It does this by phosphorylating and regulating the activity of a large number of downstream effectors. There are currently >100 suspected Akt substrates [6] and more are being discovered. A simplified schematic representation of this pathway is shown in FIG. 6 herein.

Akt is hyper-activated in the majority of high grade glioma (HGG) tumors and many other human cancers [7-11]. Many inhibitors of this pathway are under development or are in clinical trial for treatment of cancer patients [12-15]. It is not known if the pathway is used similarly among patients with a specific cancer. If different “branches” of the pathway are activated in different patients, this might determine how patients respond to targeted therapies. Since Akt is an important determinant of how cancer cells respond to chemotherapy and radiation, this applies to other anti-neoplastic and conventional therapies also.

### Example 5

### Five Tumor Subtypes are Identified Based on Expression of PI3K/Akt Pathway Genes

To investigate if the pathway is used differently among HGG patients, the inventors used expression of Akt pathway genes and clustering methods. The inventors generated a hand curated list of Akt pathway genes using PubMed literature searches and protein databases. The following categories were included: 1) upstream regulators and activators of Akt, 2) proteins that physically interact with Akt, 3) downstream effectors phosphorylated by Akt and 4) proteins in complexes known to interact with, regulate or be regulated by Akt (for example all proteins in mTORC1 and mTORC2).

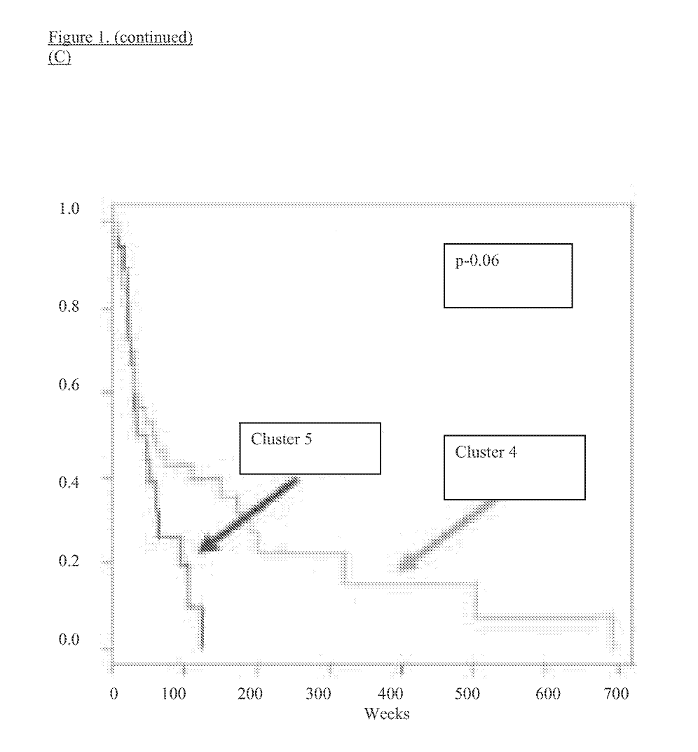

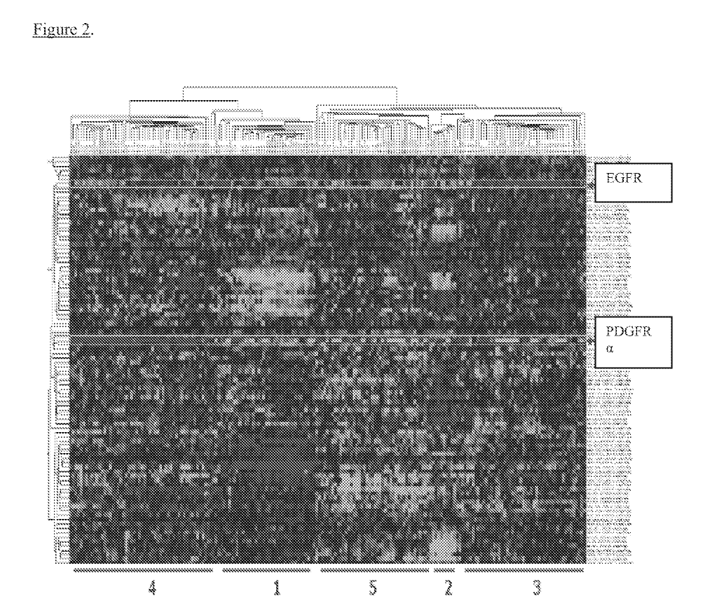

The inventors generated a correlation between clustered samples plot (FIG. 2) using the list of Akt pathway genes in a published expression profiling dataset containing 185 HGG and 14 non-neoplastic “autopsy” samples (FIG. 2A). This analysis gives information on the similarity of total Akt pathway gene expression between tumors. In FIG. 2a, tumors are plotted on both axis. If PI3K/Akt pathway genes of 2 tumors are positively correlated then the intersection of the 2 tumors is shown in red; intersection of tumors with negatively correlated Akt pathway gene expression are green; and intersections of tumors with little Akt pathway correlation are black.

This data shows that there are 6 patient subgroups that have similar expression of Akt pathway genes (clusters 1-5, FIG. 2A) and a group of patients (lower left, FIG. 2A) that have gene expression profiles with low similarity to any cluster. Subgroups are associated with different survival curves (FIG. 2B). Difference between survival for patients in clusters 4 and 5 approached statistical significance (p=0.06 log rank test; FIG. 2C). This data shows tumor subgroups exist that regulate Akt pathway genes differently, and these subgroups use different “branches” of the Akt pathway. It follows that patient subgroups will respond to pathway inhibitors differently. They may also respond differently to chemotherapy and radiation.

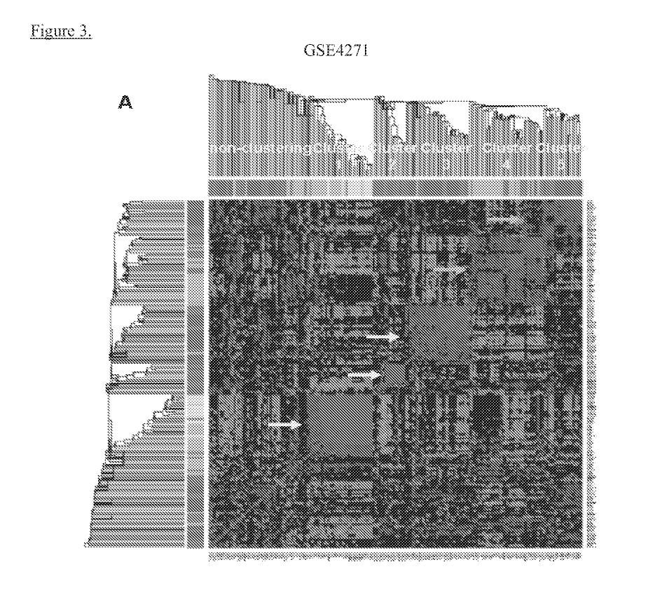

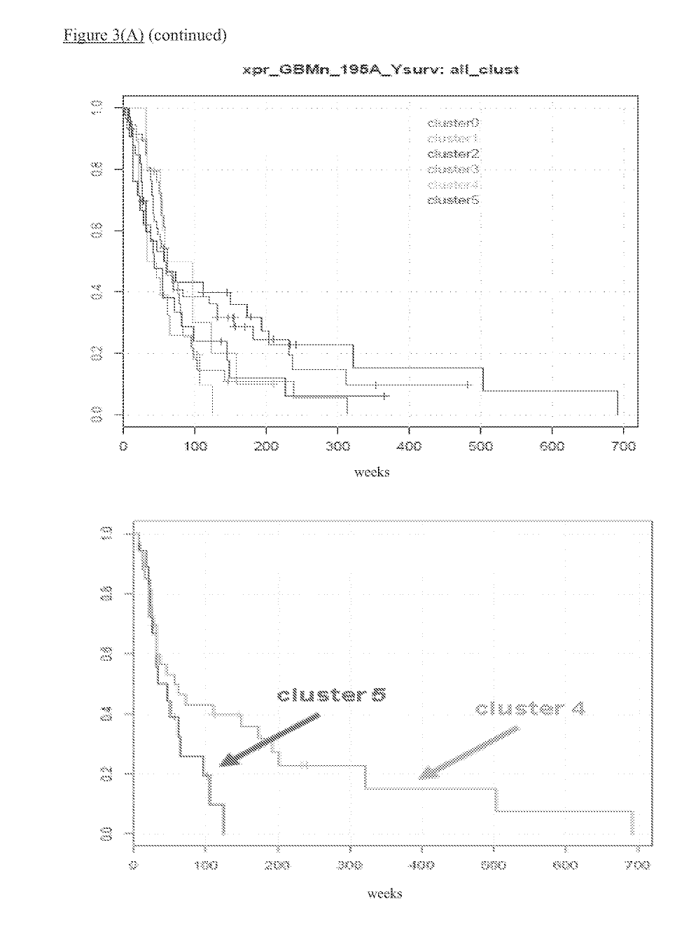

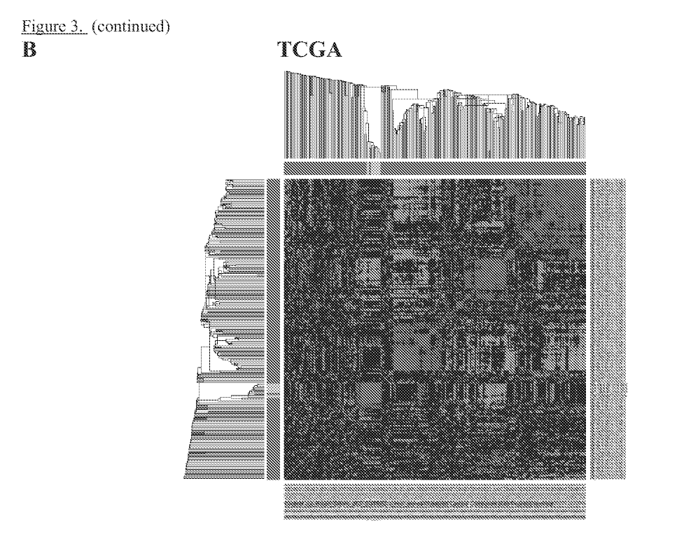

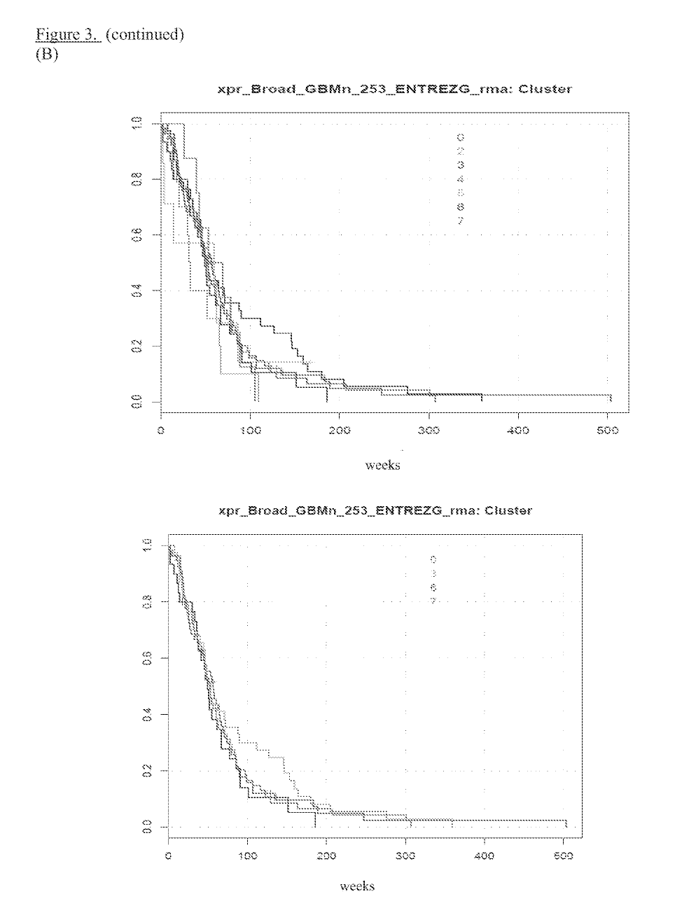

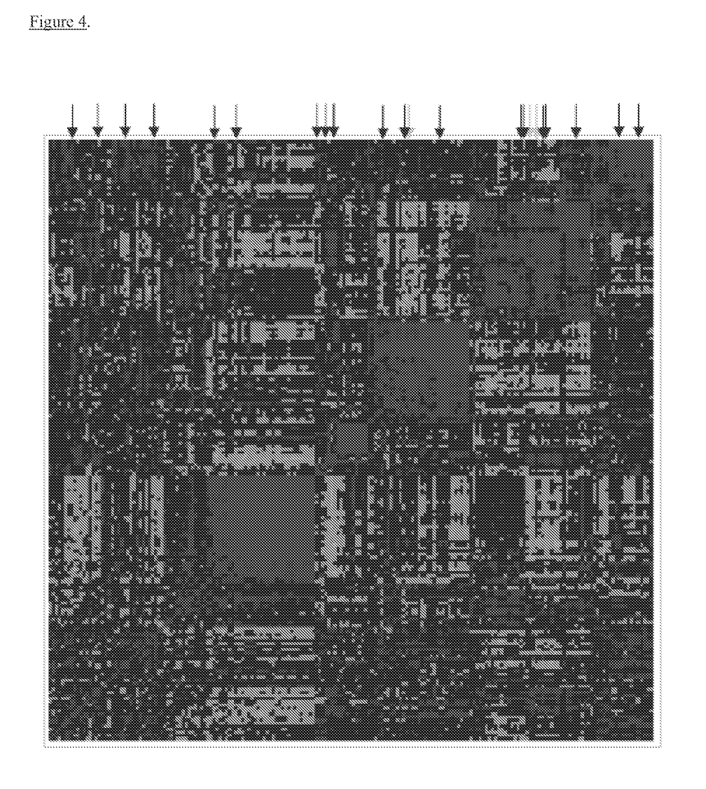

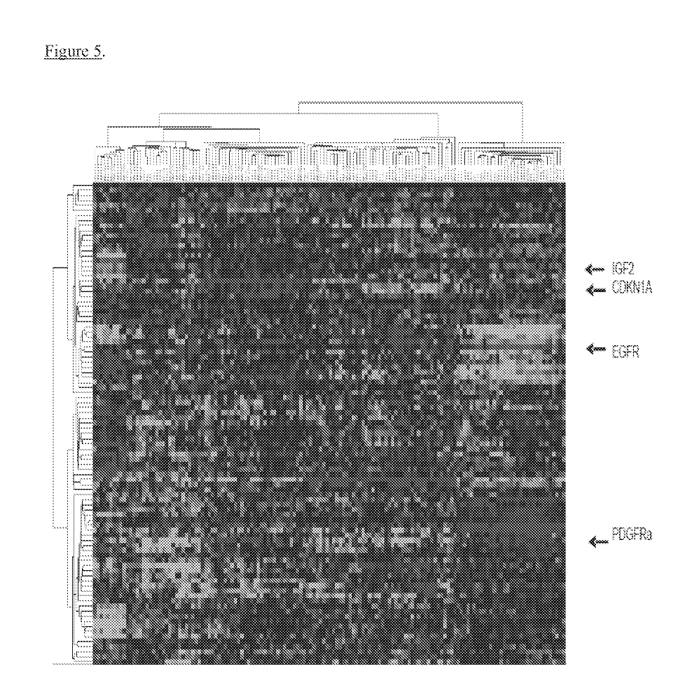

Each subgroup may be analyzed for functional categories of genes. This may be accomplished by finding genes that are expressed differently between subgroups. The inventors used an unsupervised clustering method that classifies similar objects into groups. In this case tumors with similar expression of Akt pathway genes are clustered (FIG. 3). Tumors are listed at the top and genes at the sides. If the expression of a gene is high in a tumor the intersection between gene and tumor is red; if it is low then green. These analyses should demonstrate which Akt pathway genes are important in each subgroup and therefore which inhibitors should work in specific subgroups. For example, a preliminary analysis shows that PDGFRα is overexpressed in subgroup 4 and EGFR is overexpressed in subgroup 3 (FIG. 3). The inventors believe that patients in subgroup 4 will respond to PDGFRα inhibitors and patients in subgroup 3 will respond to EGFR inhibitors. Additionally, as readily apparent to one of skill in the art, any number of other analysis maybe used to find which Akt pathway genes are important in each subgroup. These analyses can also be used to find other genes, not necessarily directly associated with the Akt pathway, that are important to the Akt subgroup.

### Example 6

### Summary

The inventors demonstrate there are 6 major subgroups of HGG that regulate Akt pathway genes differently. The inventors believe that these subgroups use different “branches” of the Akt pathway and will respond differently to conventional and targeted therapies. Therefore this analysis may be used to match therapy to patient. A potential benefit of this approach over current methods that analyze a single molecule or gene is that this analysis can allow a more comprehensive categorization of patients and selection between multiple therapy options.

### Example 7

### Significance

The inventors believe that the described analysis can be used to match therapy to patient. Subgroups may define patients that will respond to specific therapies targeting growth factors or the PI3K/Akt pathway. They can also define patients that will respond to conventional therapies. Since the PI3K/Akt pathway is important in many other cancers these results can apply to other cancers. The same type of analysis performed on other cancer-associated pathways (Ras, Notch etc . . . ) may also yield subgroups defining patients that will respond to targeted therapies against those pathways.

### Example 8

### AKT Pathway Gene Expression Divides Human-Rodent GBM Xenografts into Classes

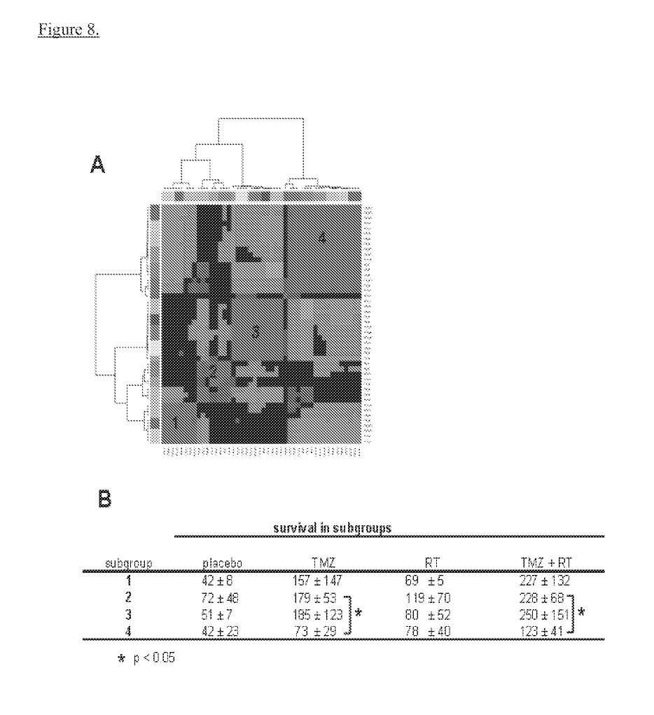

The inventors used rodent models of human Akt class to test response to human therapies find if Akt class predicts response to therapy. FIG. 8A is an Akt pathway correlation map of gene expression data from replicates of 15 rodent glioma xenografts. The analysis indicates 4 Akt xenograft classes. It is evident that xenograft models are readily classified by Akt gene expression. Distribution of biological replicates indicated by colors next to the axes demonstrates excellent Akt class stability. Similarities between Akt pathway maps demonstrate xenografts mimic gene expression of parental tumors, consistent with published reports of xenograft models of other tumors. The inventors investigate relationships between human and rodent Akt classes by mapping Akt pathway gene expression from xenografts onto human tumors. It is found 15% of genes have different expression in xenografts compared to the human tumors. When these genes are removed 6 of 7 xenografts cluster with parental human tumors. These data demonstrate human-rodent xenografts model human Akt class.

### Example 9

### Response to Therapy Depends on Akt Class in Human-Rodent Xenograft Models

In FIG. 8B, the inventors analyzed whether xenograft drug sensitivity is associated with Akt class. Xenograft group 2 is more sensitive to temozolomide (TMZ) and temozolomide plus radiation (TMZ+RT) than group 4 (p<0.05). The data demonstrates TMZ sensitivity is associated with Akt class and that Akt class predicts therapeutic response.

### Example 10

### Table 1—Possible List of Genes to Distinguish Subgroups

TABLE 1
Genetic loci and corresponding ID number

| | |
|---|---|
| SORBS | 8470 |
| PPP2R2C | 5522 |
| TP53 | 7157 |
| PIK3C3 | 5289 |
| FGFR3 | 2261 |
| PPP2R5B | 5526 |
| Akt1 | 207 |
| Akt1S1 | 84335 |
| HIF1A | 3091 |
| EIF4EBP1 | 1978 |
| EGFR | 1956 |
| PDGFC | 56034 |
| PDGFA | 5154 |
| PHLPP | 23239 |
| PDGFRA | 5156 |
| RICTOR | 253260 |
| AKT1P | 64400 |
| TWIST | 7291 |
| CCND1 | 595 |
| MDM2 | 4193 |
| GAB2 | 9846 |
| HSP90B1 | 7184 |

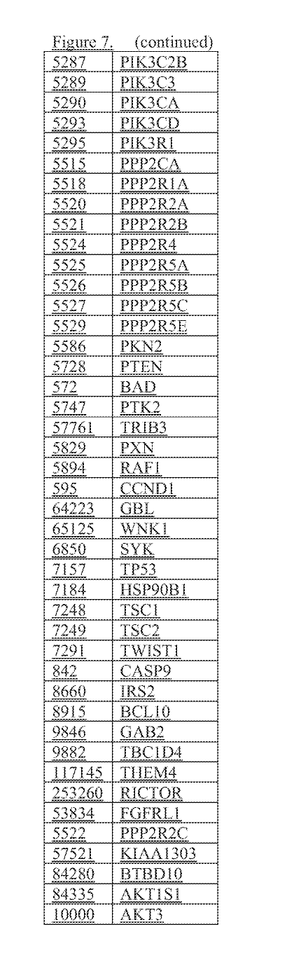

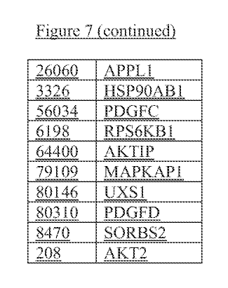

As readily apparent to one of skill in the art, any number of genetic loci and/or biomarkers could be used to subgroup tumors and conditions, and the invention is not in any way limited to those genes listed in Table 1 or FIG. 7 herein. For example, other genes related, both directly and indirectly to the Akt pathway, could be clustered and thus used for subgrouping a condition, disease and/or tumor. Or, for example, other methods of identifying Akt subgroups include the use of biomarkers that include nucleic acid(s), protein(s), modified protein(s), mutated or modified nucleic acid(s), epigenetic changes or a change(s) in DNA copy number associated with Akt subgroups. Similarly, this analysis may be generalized to any cancer that has Akt pathway activation, and the invention is in no way limited to GBM.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

### REFERENCES

1. Sinor, A. D. and L. Lillien, Akt-1 expression level regulates CNS precursors. J Neurosci, 2004. 24(39): p. 8531-41.
2. Androutsellis-Theotokis, A., et al., Notch signaling regulates stem cell numbers in vitro and in vivo. Nature, 2006. 442(7104): p. 823-6.
3. Testa, J. R. and P. N. Tsichlis, AKT signaling in normal and malignant cells. Oncogene, 2005. 24(50): p. 7391-3.
4. Bellacosa, A., et al., Activation of AKT kinases in cancer: implications for therapeutic targeting. Adv Cancer Res, 2005. 94: p. 29-86.
5. Franke, T. F., PI3K/Akt: getting it right matters. Oncogene, 2008. 27(50): p. 6473-88.
6. Manning, B. D. and L. C. Cantley, AKT/PKB signaling: navigating downstream. Cell, 2007. 129(7): p. 1261-74.
7. Chen, J. Q., et al., The Akt/PKB pathway: molecular target for cancer drug discovery. Oncogene, 2005. 24(50): p. 7482-92.
8. Alessi, D. R., et al., Molecular basis for the substrate specificity of protein kinase B; comparison with MAPKAP kinase-1 and p70 S6 kinase. FEBS Lett, 1996. 399(3): p. 333-8.
9. Haas-Kogan, D., et al., Protein kinase B (PKB/Akt) activity is elevated in glioblastoma cells due to mutation of the tumor suppressor PTEN/MMAC. Curr Biol, 1998. 8(21): p. 1195-8.
10. Holland, E. C., et al., Combined activation of Ras and Akt in neural progenitors induces glioblastoma formation in mice. Nat Genet, 2000. 25(1): p. 55-7.
11. Altomare, D. A. and J. R. Testa, Perturbations of the AKT signaling pathway in human cancer. Oncogene, 2005. 24(50): p. 7455-64.
12. Granville, C. A., et al., Handicapping the race to develop inhibitors of the phosphoinositide 3-kinase/Akt/mammalian target of rapamycin pathway. Clin Cancer Res, 2006. 12(3 Pt 1): p. 679-89.
13. Hennessy, B. T., et al., Exploiting the PI3K/AKT pathway for cancer drug discovery. Nat Rev Drug Discov, 2005. 4(12): p. 988-1004.
14. Tokunaga, E., et al., Deregulation of the Akt pathway in human cancer. Curr Cancer Drug Targets, 2008. 8(1): p. 27-36.
15. Garcia-Echeverria, C. and W. R. Sellers, Drug discovery approaches targeting the PI3K/Akt pathway in cancer. Oncogene, 2008. 27(41): p. 5511-26.
16. Li, A., et al., Unsupervised analysis of transcriptomic profiles reveals six glioma subtypes. Cancer Res, 2009. 69(5): p. 2091-9.
17. Nigro, J. M., et al., Integrated array-comparative genomic hybridization and expression array profiles identify clinically relevant molecular subtypes of glioblastoma. Cancer es, 2005. 65(5): p. 1678-86.
18. Phillips, H. S., et al., Molecular subclasses of high-grade glioma predict prognosis, elineate a pattern of disease progression, and resemble stages in neurogenesis. Cancer cell, 2006. 9(3): p. 157-73.
19. Shai, R., et al., Gene expression profiling identifies molecular subtypes of gliomas. oncogene, 2003. 22(31): p. 4918-23.
20. Liang, Y., et al., Gene expression profiling reveals molecularly and clinically distinct subtypes of glioblastoma multiforme. Proc Natl Acad Sci USA, 2005. 102(16): p. 814-9.
21 Manning, B. D. and L. C. Cantley, AKT/PKB signaling: navigating downstream. Cell, 2007. 129(7): p. 1261-74.
22. Haas-Kogan, D., et al., Protein kinase B (PKB/Akt) activity is elevated in glioblastoma cells due to mutation of the tumor suppressor PTEN/MMAC. Curr Biol, 1998. 8(21): p. 1195-8.
23. Ermoian, R. P., et al., Dysregulation of PTEN and protein kinase B is associated with glioma histology and patient survival. Clin Cancer Res, 2002. 8(5): p. 1100-6.
24. Haas-Kogan, D. A., et al., Epidermal growth factor receptor, protein kinase B/Akt, and glioma response to erlotinib. J Natl Cancer Inst, 2005. 97(12): p. 880-7.
25. Castellino, R. C. and D. L. Durden, Mechanisms of disease: the PI3K-Akt-PTEN signaling node—an intercept point for the control of angiogenesis in brain tumors. Nat Clin Pract Neurol, 2007. 3(12): p. 682-93.
26. LoPiccolo, J., et al., Targeting the PI3K/Akt/mTOR pathway: effective combinations and clinical considerations. Drug Resist Updat, 2008. 11(1-2): p. 32-50.
27. Phillips, H. S., et al., Molecular subclasses of high-grade glioma predict prognosis, delineate a pattern of disease progression, and resemble stages in neurogenesis. Cancer Cell, 2006. 9(3): p. 157-73.
28. Neale, G., et al., Molecular characterization of the pediatric preclinical testing panel. Clin Cancer Res, 2008. 14(14): p. 4572-83.
29. Whiteford, C. C., et al., Credentialing preclinical pediatric xenograft models using gene expression and tissue microarray analysis. Cancer Res, 2007. 67(1): p. 32-40.

1. A method of diagnosing a cancer subtype in an individual, comprising:
determining the presence or absence of an abnormal expression of an Akt pathway gene cluster in the individual; and
diagnosing the cancer subtype based on the presence of the abnormal expression of the Akt pathway gene cluster in the individual.

2. The method of claim 1, wherein the cancer is glioblastoma multiforme (GBM).

3. The method of claim 1, wherein the Akt pathway gene cluster is generated from one or more genetic loci listed in FIG. 7 herein.

4. The method of claim 1, wherein the abnormal expression of an Akt pathway gene cluster comprises an overexpression of PDGFR\acute{α} and/or EGFR in the individual.

5. The method of claim 1, wherein the individual is a human.

6. The method of claim 1, wherein the individual is a mouse and/or rat.

7. The method of claim 1, wherein the Akt pathway gene cluster comprises one or more of the following genetic loci: SORBS, PPP2R2C, TP53, PIK3C3, FGFR3, PPP2R5B, Akt1, Akt1S1, HIF1A, EIF4EBP1, EGFR, PDGFC, PDGFA, PHLPP, PDGFRA, RICTOR, AKT1P, TWIST, CCND1, MDM2, GAB2 and/or HSP90B1.

8. The method of claim 1, wherein the abnormal expression of the Akt pathway gene cluster comprises a high level of expression relative to a normal subject of SORBS, PPP2R2C, TP53, PIK3C3, FGFR3, PPP2R5B, Akt1, Akt1S1, HIF1A, EIF4EBP1, EGFR, PDGFC, PDGFA, PHLPP, PDGFRA, RICTOR, AKT1P, TWIST, CCND1, MDM2, GAB2 and HSP90B1, or any combinations thereof.

9. A method of treating cancer in an individual, comprising:
diagnosing a cancer subtype in the individual based on a cluster of Akt pathway gene expression; and
treating the individual.

10. The method of claim 9, wherein the cluster of Akt pathway gene expression is generated from one or more genetic loci listed in FIG. 7 herein.

11. The method of claim 9, wherein treating the individual comprises administering a therapeutically effective dosage of temodar (TMZ) to the individual.

12. The method of claim 9, wherein treating the individual comprises administering a therapeutically effective dosage of PDGFR\acute{α} inhibitor to the individual.

13. The method of claim 9, wherein treating the individual comprises administering a therapeutically effective dosage of EGFR inhibitor to the individual.

14. The method of claim 9, wherein the cluster of Akt pathway gene expression comprises one or more of the following genetic loci: SORBS, PPP2R2C, TP53, PIK3C3, FGFR3, PPP2R5B, Akt1, Akt1S1, HIF1A, EIF4EBP1, EGFR, PDGFC, PDGFA, PHLPP, PDGFRA, RICTOR, AKT1P, TWIST, CCND1, MDM2, GAB2 and/or HSP90B1.

15. The method of claim 9, wherein the cluster of Akt pathway gene expression comprises a high level of expression relative to a normal subject of SORBS, PPP2R2C, TP53, PIK3C3, FGFR3, PPP2R5B, Akt1, Akt1S1, HIF1A, EIF4EBP1, EGFR, PDGFC, PDGFA, PHLPP, PDGFRA, RICTOR, AKT1P, TWIST, CCND1, MDM2, GAB2 and HSP90B1, or any combinations thereof.

16. The method of claim 9, wherein diagnosing the cancer subtype based on the cluster of Akt pathway gene expression comprises protein analysis, polypeptide modification, polynucleotide modification, gene mutation analysis, and/or gene sequencing.

17. The method of claim 9, wherein the cancer is glioblastoma multiforme (GBM).

18. A method of diagnosing a tumor subtype, comprising:
obtaining a tumor sample from an individual;
assaying the tumor sample to determine the presence or absence of an abnormal expression of an Akt pathway gene cluster; and
diagnosing the tumor subtype based on the presence of the abnormal expression of the Akt pathway gene cluster.

19. The method of claim 18, wherein the abnormal expression of the Akt pathway gene cluster comprises a high level of expression relative to a normal subject of SORBS, PPP2R2C, TP53, PIK3C3, FGFR3, PPP2R5B, Akt1, Akt1S1, HIF1A, EIF4EBP1, EGFR, PDGFC, PDGFA, PHLPP, PDGFRA, RICTOR, AKT1P, TWIST, CCND1, MDM2, GAB2 and HSP90B1, or any combinations thereof.

20. The method of claim 18, wherein the tumor comprises glioblastoma multiforme (GBM).

21. The method of claim 18, wherein the Akt pathway gene cluster comprises any biomarker including but not limited to nucleic acids, proteins, modified proteins, mutated or modified nucleic acids, epigenetic changes or an associated change in DNA copy number.
